(19) United States
(12) Patent Application Publication
(10) Pub. No.: US 2020/0301679 A1
(43) Pub. Date: Sep. 24, 2020

(54) SYSTEM FOR CREATING MOBILE AND WEB APPLICATIONS FROM A GRAPHICAL WORKFLOW SPECIFICATION

(71) Applicant: SNAPIOT INC, Rancho Santa Fe, CA (US)

(72) Inventors: Isaac Eshagh Eteminan, Rancho Santa Fe, CA (US); James William Bishop, JR., Reno, NV (US); Marco Carosi, Roma (IT); Alessandro Salimbeni, Rome (IT)

(21) Appl. No.: 16/899,188

(22) Filed: Jun. 11, 2020

Related U.S. Application Data
(63) Continuation-in-part of application No. 16/778,665, filed on Jan. 31, 2020.
Application No. 16/778,665 is a continuation-in-part of application No. 16/805,683, filed on Feb. 28, 2020.
Application No. 16/805,683 is a continuation-in-part of application No. 16/100,078, filed on Aug. 9, 2018.

(51) Int. Cl. G06F 8/34; G06Q 10/10; G16H 10/20; G16H 40/20; G06F 8/30; G06F 11/36; G06F 8/41; G06F 8/60; G09B 19/00

(52) U.S. Cl. CPC G06F 8/34; G06Q 10/103; G06Q 10/101; G16H 10/20; G16H 40/20; G06F 3/0482; G06F 11/3688; G06F 11/3664; G06F 8/41; G06F 8/60; G09B 19/0053; G06F 8/31

(57) ABSTRACT

An Application Factory system and method with a Composition Studio, where a workflow for a Device Application or Component is generated from user-initiated selections of at least one of a logic and data element from an Event, Logic, Data, Math, Workflow, or Service Palette, and a user interaction element from a Graphic, Widget, Audio/Video, or Haptic Palette. The workflow is displayed on a Data and Logic Canvas or Multimedia User Interaction Canvas of a user device. A Build Engine supports compilation of the Device Application or Component. And a Generic Device Model provides testing of the Device Application or Component. A Distribution Center provides at least one of discovery of, education on, demonstration of, experimentation with, and acquisition of the Device Application or Component. The Composition Studio utilizes a Visual and XML-Based Representation Language to enable the workflow to be generated without the user having to perform software coding.

### CROSS-REFERENCE TO RELATED APPLICATIONS

This application is a continuation-in-part of, and claims the benefit and priority to: U.S. patent application Ser. No. 16/100,078, titled “Mobile-Native Clinical Trial Operations,” filed Aug. 9, 2018; U.S. patent application Ser. No. 16/100,094, titled “Mobile-Native Clinical Trial Operations Service Suite,” filed Aug. 9, 2018; U.S. patent application Ser. No. 16/778,665, titled “Offline Mode in a Mobile-Native Clinical Trial Operations System,” filed Jan. 31, 2020; U.S. patent application Ser. No. 16/805,683, titled “Offline Mode in a Mobile-Native Clinical Trial Operations Service Suite,” filed Feb. 28, 2020; and U.S. patent application Ser. No. 16/818,634, titled “Automatic Self-Documentation in a Mobile-Native Clinical Trial Operations System and Service Suite,” filed Mar. 13, 2020, the contents of which are incorporated herein by reference in their entirety.

### FIELD

This invention relates to automatic code generation for web and mobile device applications using a workflow-description language as the starting point. It is described here in the context of information technology that supports clinical trial operations, but it may be applied broadly to other contexts that can benefit from rapid development of applications supporting business processes.

### BACKGROUND

U.S. Pat. No. 8,719,776 describes a system, hereinafter called an Application Factory, the subject matter of which is incorporated by reference herein, for creating and distributing mobile device applications from a specification expressed in a graphical programming language. U.S. patent application Ser. Nos. 16/100,078 and 16/100,094 apply an embodiment(s) of that system as a portion of a larger system, hereinafter called SnapClinical, aimed at supporting clinical trial operations by providing rapid customization of software applications that implement and enforce a trial's science and ethics protocols. U.S. patent application Ser. Nos. 16/778,665, 16/805,683 and 16/818,634 provide further enhancements to SnapClinical.

A foundational technology mentioned and partially described in the SnapClinical disclosures is the addition of a workflow language to the Application Factory, which allows a subject-matter expert who is not also a software engineer to express a set of business processes graphically then generate automatically the corresponding HTML5-based web application (both server and client/browser code) and mobile device native applications for multiple platforms (such as IOS and Android). The present disclosure aims to explicitly address and provide additional details for this workflow capability.

### SUMMARY

The following presents a simplified summary in order to provide a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented later.

In one aspect of the disclosed embodiments, an Application Factory system containing computer-executable instructions is provided, comprising: a Composition Studio, where a workflow for at least one of a Device Application and a Device Application Component is generated from a series of user-initiated selections of at least one of a logic and data element from an Event, Logic, Data, Math, Workflow, or Service Palette, and a user interaction element from a Graphic, Widget, Audio/Video, or Haptic Palette, the workflow being displayed on at least one of a Data and Logic Canvas and Multimedia User Interaction Canvas of a user device; a Build Engine supporting a compilation of the Device Application or Device Application Component; a Generic Device Model providing a test of the Device Application or Device Application Component; and a Distribution Center, running on a server, providing at least one of discovery of, education on, demonstration of, experimentation with, and acquisition of the Device Application or Device Application Component, wherein the Composition Studio utilizes a Visual and XML-Based Representation Language to enable the workflow to be generated without the user having to perform software coding.

In another aspect of the disclosed embodiments, the above system is provided further comprising a Verification Engine providing step level simulation and testing of the Device Application or Device Application Component; and/or further comprising a selection option in the Composition Studio, the option enabling the Build Engine to compile the Device Application or Device Application Component for operation on either a mobile device or web browser; and/or wherein the workflow is a clinical trial workflow; and/or further comprising a computer running the Application Factory system's computer-executable instructions; and/or further comprising an Internet connection between the user device and the Distribution Center's server; and/or further comprising an Internet connection between the mobile device or web browser and the Distribution Center's server.

In yet another aspect of the disclosed embodiments, a method for building and distributing a device software application without software coding is provided, comprising computer-implemented steps of: generating a workflow for at least one of a Device Application and a Device Application Component from a series of user-initiated selections of at least one of a logic and data element from an Event, Logic, Data, Math, Workflow, or Service Palette, and a user interaction element from a Graphic, Widget, Audio/Video, or Haptic Palette; displaying the workflow on at least one of a Data and Logic Canvas and Multimedia User Interaction Canvas on a user device; compiling the Device Application or Device Application Component for operation on at least one of a mobile device and web browser; testing of the Device Application or Device Application Component; and uploading the Device Application or Device Application Component to a Distribution Center, wherein the Device Application or Device Application Component can be run on the mobile device or web browser, wherein a Visual and XML-Based Representation Language is used to enable the workflow to be generated without performing software coding.

In yet another aspect of the disclosed embodiments, the above method is provided, further comprising simulating and testing of the Device Application or Device Application Component at a step level; and/or further offering a selection option to compile the Device Application or Device Application Component for operation on either a mobile device or web browser; and/or wherein the generated workflow is a clinical trial workflow.

### BRIEF DESCRIPTION OF THE DRAWINGS

FIG. 1 illustrates an exemplary schematic block diagram of an Application Factory system.

FIG. 2 illustrates an exemplary schematic block diagram of a Composition Studio in an Application Factory system embodiment.

FIG. 3 illustrates workflow and user interface language elements as may be supported by an Application Factory system embodiment.

FIG. 4 illustrates an exemplary workflow which may be included in a web or mobile device application by an Application Factory system embodiment.

FIG. 5 illustrates an exemplary Composition Studio canvas and palette layout for workflow development as may be supported by an Application Factory system embodiment.

FIG. 6 illustrates an exemplary Composition Studio canvas and palette layout for user interface development as may be supported by an Application Factory system embodiment.

FIG. 7 illustrates a schematic block diagram of an exemplary computer system used to implement the exemplary embodiments.

### DETAILED DESCRIPTION

The following detailed description describes currently contemplated modes of carrying out exemplary embodiments. The description is not to be taken in a limiting sense but is made merely for the purpose of illustrating the general principles of some embodiments. Therefore, based on the description, various modifications and changes may be made by one of ordinary skill in the art to devise alternative embodiments that perform or accomplish similar results, and such modifications and changes are understood to be within the spirit and scope of this disclosure.

Various features are described below that can each be used independently of one another or in combination with other features.

Numerous enhancements have been made to the Application Factory system since its initial disclosure in U.S. Pat. No. 8,719,776, which is incorporated herein by reference. Two key enhancements are detailed here.

First, the Build Engine is now able to create not only native mobile applications for deployment to all existing mobile device types as previously disclosed, but also web applications for deployment to a web browser via a web server. Each application may be deployed as either a web application or a mobile application, or both, at the discretion of the developer using the Application Factory system.

Second, under the umbrella described in the original disclosure as “enhanced concepts” incorporated in the Data and Logic Canvas at “higher . . . Levels” of “complexity or capability,” the Visual Representation Language has been extended to incorporate multiple additional modes of expression covering multiple additional types of logic and data structure. The Composition Studio is now able to support these new language modes, and the Build Engine is now able to incorporate corresponding code, into generated applications, services, and data structures. The new visual language construct of interest in the present disclosure is the Business Process Model and Notation (BPMN), a standard decision-tree or workflow language used by vertical-industry subject-matter experts who are not necessarily software engineers to model their business activities. The Application Factory system's Composition Studio provides a palette of BPMN model elements and a workflow canvas on which to arrange them, while its Build Engine provides automatic translation of BPMN workflows into its internal XML-Based Representation Language and thence to compiled native mobile device (IOS, Android, etc.) and web (HTML5) applications.

While BPMN is used as the workflow language for the exemplary embodiments, it is understood that BPMN is simply an example of one of several possible languages that can achieve the desired results. BPMN was adopted due to its standardization and relative flexibility of use. Accordingly, other similar functioning languages may be used, according to design preference.

Therefore, what had previously been the exclusive realm of software engineers or programmers, requiring line by line coding of software to create the necessary or desired user interfaces with inputs and outputs, has now become achievable by a non-programmer.

Specifically, a technical result (an Application or Application Component) can be arrived at with no line by line coding by the designer. Therefore, the invention reduces the need for long development cycles (with software engineers/programmers) and allows an end designer to develop his desired product in hours or days, versus the typical weeks or months. In the context of SnapClinical as a medical trial operation, the exemplary invention can be used to rapidly generate a clinical trial software platform. Further, by automating the described processes, development costs can be significantly reduced.

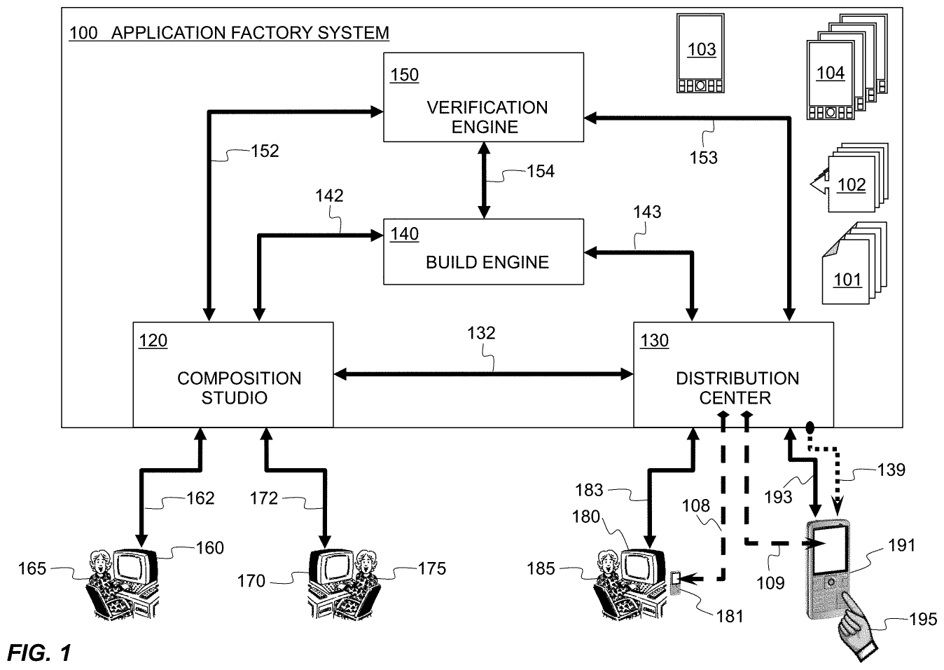

The high-level diagram of FIG. 1 depicts the primary elements of an exemplary embodiment, including the main system, its major subsystems and primary internal elements, and the primary external elements with which it interacts. In the diagram, Application Factory System 100 embodies the functionality as outlined in the Introduction above and detailed in the subsystem descriptions that follow. The functions of the Application Factory System 100 are allocated among four major subsystems, with interactions among the four major subsystems as shown. In addition, four primary internal elements are shown, which embody the reasons for the existence of Application Factory System 100.

The first primary internal element is the set of one or more Device Applications 101 which are produced, managed, and distributed using Application Factory System 100. Each Application 101 is a package of logic and data created by a developer to perform one or more particular functions upon execution by a device. By way of example, but without limitation, an Application 101 may display a sequence of images, take a photograph or capture a location and share it with others, or play a complex game involving one or more people. Note that while FIG. 1 depicts just four Applications 101, in various embodiments the actual number may be unlimited. It is expressly understood here that the device wherein the Application 101 is implemented upon, may be a mobile or non-mobile device or a web browser.

In addition to Device Applications 101, developers may create a set of one or more Application Components 102. Each Application Component 102 is a package of logic and data encapsulating a set of related functionality that may be useful in numerous Applications 101. In general Application Components 102 do not stand alone, but may be combined with additional logic and perhaps other Application Components 102 in order to form an Application 101. Note that while FIG. 1 depicts just four Application Components 102, in various embodiments the actual number may be unlimited.

Application Factory System 100 incorporates a single abstraction of a device that all Applications 101 and Application Components 102 will execute on. This Generic Device Model 103 is used as a common platform for development, testing, simulation, and demonstration of any Application 101. Having a common target ensures consistency of performance across multiple real target devices and multiple Applications 101, and simplifies the learning process for novice developers. Generic Device Model 103 embodies a superset of the features and capabilities found in real devices that are well known to those skilled in the art, including without limitation at least one of modules for wireless connectivity with a cellular network, Wifi hot spots, and Bluetooth devices; support for communication modes such as signaling, circuit calls, and packet transmission and reception; ports for wired connectivity with nearby devices such as a headphone jack and a USB connector; various human interface devices including primary and optional secondary video display screens, a numeric or alphabetic keypad that may be optionally backlit, miscellaneous buttons that activate functions such as speaker volume control and others, a touchpad or multitouch gesture input pad, speakers, and microphones; environment sensors and effectors such as an accelerometer or motion and orientation sensor, a camera or light sensor, a Global Positioning System (GPS) receiver or other location sensor, a mechanical vibration generator or other haptic effector, and a thermometer or other temperature sensor. Generic Device Model 103 features a common operating system and application programming interface supporting access to all included capabilities by Applications 101.

While Generic Device Model 103 supports commonality of feature behavior across multiple Applications 101, it is also desirable for Application Factory System 100 to support the specific hardware and software associated with real mobile and non-mobile devices, so that developers may ensure their Applications 101 and Application Components 102 work in one or more actual devices, or even to target development specifically to only one actual device. To that end, Application Factory System 100 incorporates as the fourth and final primary internal element an extensible set of one or more Specific Device Models 104, each of which embodies the specific features and capabilities of a real mobile device or web platform/device type. To the extent possible each Specific Device Model 104 is a specialization of Generic Device Model 103, but in general platform details such as the real software operating system—Symbian, Windows, Linux, WebOS, IOS, Android, MacOS, and so forth—and real device programming interfaces are included. As well, visual elements such as a graphical representation of the actual device, and behavior elements such as an execution simulator, are provided in each Specific Device Model 104. In an exemplary embodiment, most aspects of a Specific Device Model 104 are incorporated in Application Factory System 100 by embedding or providing programmatic access to the features of the real device's or platform's actual software development toolkit, thereby ensuring the most accurate possible representation.

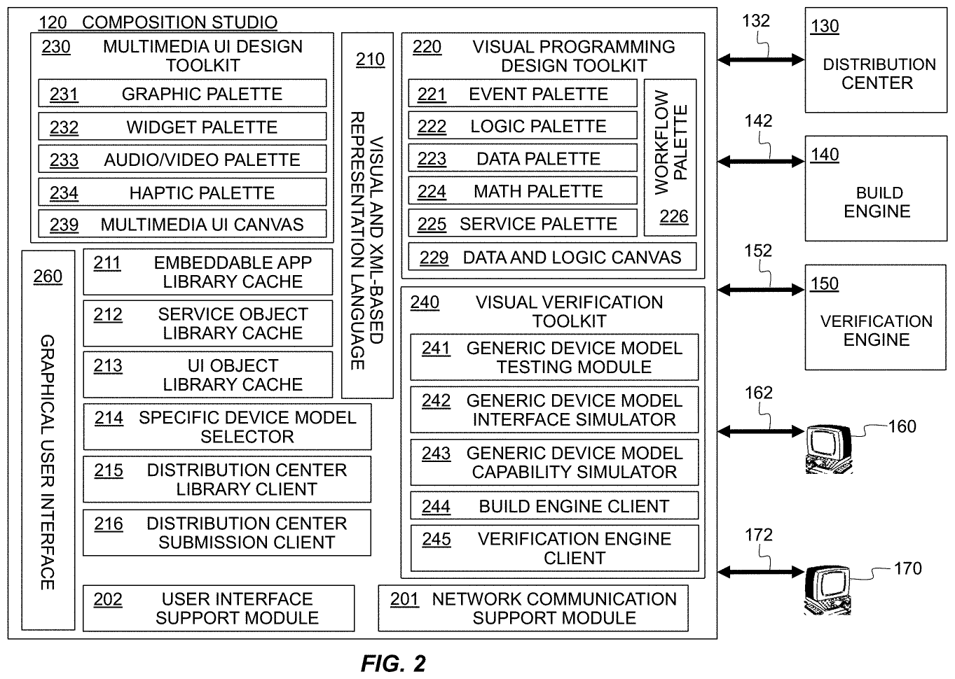

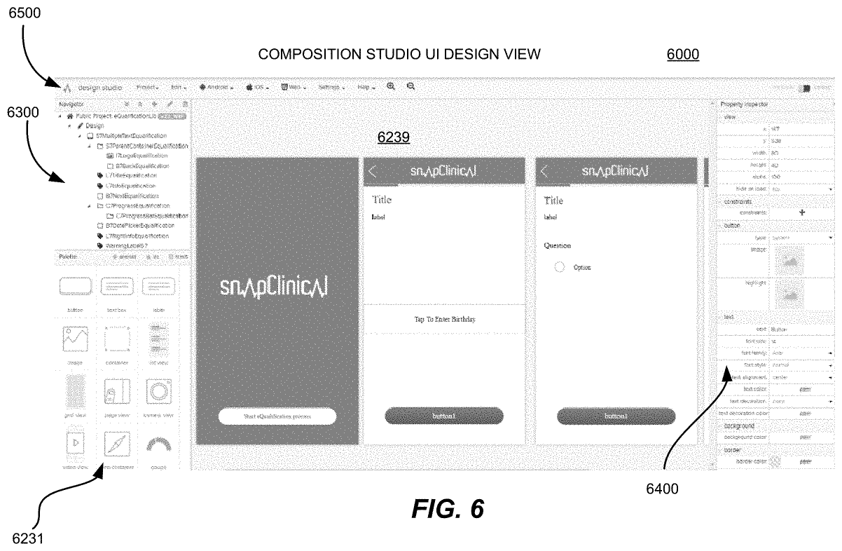

The first major subsystem, Composition Studio 120, embodies the functions associated with a development environment, providing the ability to develop Applications 101 and Application Components 102. Composition Studio 120 may support multiple programming paradigms, natively including at least a novice mode suitable for Novice Developer 165 in which features are constrained and logical elements are represented visually, and an expert mode suitable for Expert Developer 175 in which all features are available and logical elements are represented textually. Another mode may support the BPMN-based workflow language that is a subject of this disclosure, providing a visual representation suitable for developers who might be called “expert novices”—expert with respect to a particular domain and novice with respect to software development. Modes may be accessed and utilized flexibly through toggling preference items that enable different feature sets, or as default configurations associated with specific developer identities or embodiments. For example, the SnapClinical embodiment may enable only the workflow mode. Additional detail on the features and presentation of different programming modes is provided in FIGS. 2-6 below and also in U.S. Pat. No. 8,719,776's FIG. 6 and attendant description, as well as in the incorporated applications listed above.

A user of Composition Studio 120 accesses its capabilities using a computer workstation or personal computer, for example, as is typical of such tools and well-known to those skilled in the art. In FIG. 1, Novice Developer 165 employs Computer 160 for this purpose, while Expert Developer 175 employs Computer 170 similarly. In either case, the actual implementation of Composition Studio 120 is itself a software program executing in a centralized server computer and presents its user interface to Novice Developer 165 and Expert Developer 175 through any of several standard and well-known web browser programs running in, respectively, Computers 160 and 170. In some embodiments, the server software may run on one or more of Computers 160, 170, obviating the need for a “server-client” configuration. The interactions between Composition Studio 120 and Computers 160 and 170 take place using standard protocols, well known to those skilled in the art, such as IP, HTTP, and HTML, represented in FIG. 1 by Network Connections 162 and 172 respectively. More detail regarding Composition Studio 120 is provided in the description of FIG. 2 below.

The second major subsystem, Distribution Center 130, embodies the functions associated with an application repository, often called an “app store” or equivalent in common usage. Distribution Center 130 supports discovery of, education on, demonstration of, experimentation with, and finally acquisition of Applications 101 and Application Components 102, for any of the Specific Device Models 104 or target platforms supported by Application Factory System 100. Distribution Center 130 provides the aforementioned capabilities to customers, represented in FIG. 1 by Internet Customer 185 and Mobile Customer 195.

Internet Customer 185 accesses the capabilities of Distribution Center 130 using a computer workstation or personal computer or device, as is typical of “app stores” and well-known to those skilled in the art, employing, as an example, Computer 180 for this purpose. The actual implementation of Distribution Center 130 is itself a software program that can be executing in a centralized server computer that presents its user interface to Internet Customer 185 through any of several standard and well-known web browser programs running in Computer 180. The interactions between Distribution Center 130 and Computer 180 take place using standard protocols, well known to those skilled in the art, such as IP, HTTP, and HTML, etc. represented in FIG. 1 by Network Connection 183. Upon selecting an Application 101 for acquisition, a copy of said selected Application 101 is transferred into Device 181 belonging to Internet Customer 185 using Application Download 108. In an exemplary embodiment, Application Download 108 occurs directly into Device 181 via the wireless network (if mobile) to which Device 181 normally attaches, using over-the-air transmission techniques well known to those skilled in the art. Alternatively, Application Download 108 may occur over Network Connection 183 into Computer 180, and thence under the control of Internet Customer 185 into Device 181 via a wired connection and driver software, neither of which is shown in FIG. 1, that are typical of most device models and well known to those skilled in the art.

Mobile Customer 195 accesses the capabilities of Distribution Center 130 directly using Device 191, and if the Device 191 is a mobile device, then employing either of two techniques typical of “app stores” and well known to those skilled in the art, both of which provide equivalent functionality as previously described, including ultimately the acquisition of Applications 101 using Application Download 109. Application Download 109 is similar to Application Download 108; it may occur directly into Device 191 via the associated wireless network using over-the-air transmission, or via an inferred Computer 190 that is not shown in FIG. 1. In the first “app store” technique, Mobile Customer 195 activates a general-purpose web browser software program similar to that used by Internet Customer 185, which in turn interacts with Distribution Center 130 via Network Connection 193 using the same standard protocols as those found in Network Connection 183. In the second technique, a purpose-built software program dedicated to accessing Distribution Center 130 and itself effectively implementing the user interface aspects of Distribution Center 130 directly, is installed in Device 191 as Embedment 139. Embedment 139 is typically implemented upon manufacture of Device 191 as a portion of its embedded operating software. Alternatively, Embedment 139 may occur as a specific Application 101 acquired from Distribution Center 130 using Network Connection 193 and downloaded using Application Download 109. In an exemplary embodiment, both “app store” techniques are provided and both implementations of Embedment 139 are supported.

In addition to supporting ordinary customers such as Internet Customer 185 and Mobile Customer 195 via their respective connections as described above, Distribution Center 130 provides the aforementioned capabilities to application developer customers, such as Novice Developer 165 and Expert Developer 175, for the purpose of embedding existing Applications 101 or Application Components 102 in their own new Applications 101 or Application Components 102. Interaction 132 between Distribution Center 130 and Composition Studio 120 can provide the mechanism whereby developers utilize Distribution Center 130 through Composition Studio 120 for this purpose. Interaction 132 also allows a developer using Composition Studio 120 to offer a completed Application 101 or Application Component 102 for distribution via Distribution Center 130.

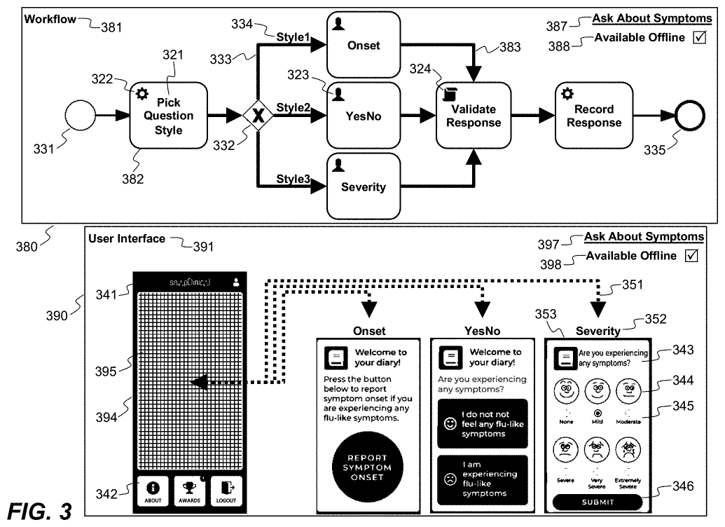

More detail regarding Distribution Center 130 is provided in U.S. Pat. No. 8,719,776's FIG. 3 and attendant description, as well as in the incorporated applications listed above.

The third major subsystem, Build Engine 140, provides compilation and linking services to the other subsystems. Build Engine 140 takes a source code package created by Composition Studio 120 or stored in Distribution Center 130, and turns the source code into executable objects embodying a particular Application 101 or Application Component 102 for the Generic Device Model 103 and the supported Specific Device Models 104 including web platforms.

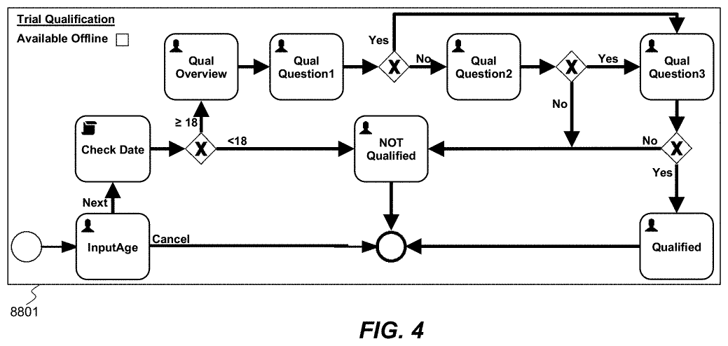

The actual implementation of Build Engine 140 is itself a software program executing, for example, in a centralized server computer and operates upon request from its various peers, according to protocols built upon standard techniques well known to those skilled in the art. Service Interaction 142 allows Composition Studio 120 to request build services of Build Engine 140, and receive the resulting objects, during the application development process. Service Interaction 143 allows Distribution Center 130 to build a specific Application 101 for a specific Device 181 or Device 191, which conforms to one of the supported Specific Device Models 104, but for which Distribution Center 130 does not have an executable version; this supports situations in which a developer provides builds of Application 101 for only a subset of Specific Device Models 104, or in which a new Specific Device Model 104 comes into existence after creation and initial distribution of Application 101. More detail regarding Build Engine 140 is provided in U.S. Pat. No. 8,719,776's FIG. 4 and attendant description, as well as in the incorporated applications listed above.

The fourth and final major subsystem of Application Factory System 100 is Verification Engine 150, which provides device simulation and testing capabilities used by developers and the other subsystems. During development of an Application 101 or Application Component 102, Novice Developer 165 or Expert Developer 175 will reach a point at which simulated execution of the developed software is appropriate in order to ascertain proper behavior. Verification Engine 150 presents a user interface by which the developer may step through the various inputs, computations, and outputs of the application and examine the results, much as a traditional software developer might do with a traditional debugger. In an exemplary embodiment, simulations for both Generic Device Model 103 and multiple Specific Device Models 104 (including web platforms/devices) are provided, allowing the application developer not only to verify correct execution on the widest possible selection of supported Specific Device Models 104, but also to assure a high probability of success on other Specific Device Models 104 that may be added to Application Factory System 100 at a later date through verification on the Generic Device Model 103. Further, in an exemplary embodiment Verification Engine 150 also provides a library of test cases suitable for use in testing various classes of Applications 101 and Application Components 102, as well as an automated testing environment in which multiple test cases can be selected and executed without developer interaction.

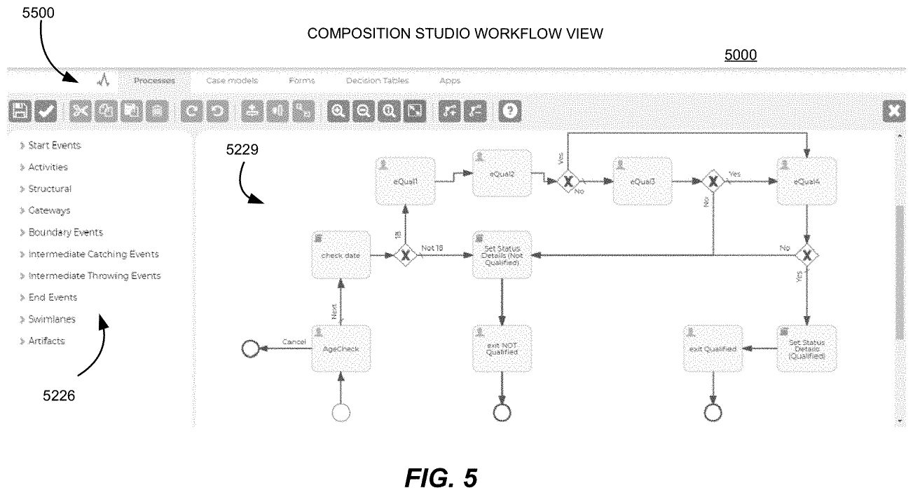

The actual implementation of Verification Engine 150 is itself a software program executing, for example, in a centralized server computer and operates upon request from its various peers, according to protocols built upon standard techniques well known to those skilled in the art. Service Interaction 152 allows Composition Studio 120 to operate testing and simulation services of Verification Engine 150 interactively during the application development process. Service Interaction 153 allows Distribution Center 130 to request automatic verification of a specific Application 101 for a specific Device 181 or Mobile Device 191, which conforms to one of the supported Specific Device Models 104, but for which Distribution Center 130 does not have a verified version; this supports situations in which a developer provides verified builds of Application 101 for only a subset of Specific Device Models 104, or in which a new Specific Device Model 104 comes into existence after creation and initial distribution of Application 101. Similarly, Service Interaction 154 allows Build Engine 140 to request automatic verification of a specific Application 101 for multiple Specific Device Models 104. This supports a scenario or use case in which a developer tests Application 101 only against the Generic Device Model 103 or a single Specific Device Model 104, then submits a bulk build request for the remainder of supported Specific Device Models 104; Build Engine 140 would in turn request bulk automatic verification of Application 101 for all supported Specific Device Models 104. More detail regarding Verification Engine 150 is provided in U.S. Pat. No. 8,719,776's FIG. 5 and attendant description, as well as in the incorporated applications listed above.

FIG. 2 depicts Composition Studio 120 in more detail, showing the major functional modules that embody the functionality outlined in the FIG. 1 description. As in FIG. 1, the significant external interfaces and corresponding cooperating elements outside Composition Studio 120 are also shown. The Distribution Center 130, Build Engine 140, Verification Engine 150, Service Interactions 132, 142, and 152, Computers 160 and 170, and Network Connections 162 and 172, shown in FIG. 2 are substantially as already described in the context of FIG. 1.

Starting at the bottom of the diagram of FIG. 2 are fundamental modules typically found in any modern software program. Network Communication Support Module 201 provides connectivity with other subsystems of Application Factory System 100, and contains such common and well known components as an inter-process message-passing software bus, message structure parsing toolkits, a TCP/IP protocol stack implementation, and networking hardware drivers appropriate for the computer hardware on which Composition Studio 120 runs. User Interface Support Module 202 provides tools for presenting information visually to a user of Composition Studio 120, such as Novice Developer 165. In an exemplary embodiment, User Interface Support Module 202 takes the form of a portable and generic application programming interface (API), well known to those skilled in the art, by which the other software components of Composition Studio 120 may create visual elements such as text, lines, polygons, animations, and so forth. Those skilled in the art will notice that Network Communication Support Module 201 and User Interface Support Module 202 are but a subset of the capabilities found in typical computer operating system software.

Fundamental to Composition Studio 120, as an environment for creation of Applications 101, is the Visual and XML-Based Representation Language 210. This Visual and)(MIL-Based Representation 210 is the language in which developers will express the logical, structural, temporal, graphical, and other elements of their ideas for an Application 101 or Application Component 102. A visual representation is provided for those, such as Novice Developer 165, who prefer or understand better via such a representation. A formal textual representation, formulated using the well known Extensible Markup Language (XML), is provided both for those, such as Expert Developer 175, who prefer or understand better via such a representation, and for use by the software modules of Composition Studio 120 and its peer subsystems in algorithmically transforming the developer's expressed ideas into a tested Mobile Device Application 101 or Device Application Component 102.

The specific visual representation is a flexible item, evolving over time as developers interact with Composition Studio 120 and provide feedback regarding the effectiveness of Language 210. Similarly, the specific XML-based textual representation is flexible as well, evolving alongside the visual representation and adapting to match it.

In an exemplary embodiment, the visual representation part of Language 210 may incorporate elements from multiple existing visual programming languages, such as BPMN, Scratch, Alice, Mindstorms, LabView, and VPL, etc. as well as potentially novel elements and elements borrowed from unrelated domains. As developer feedback drives tuning of the visual representation, it may incorporate additional elements not present in any of those progenitors.

The XML-based textual representation part of Language 210 is generally readily derivable from the visual representation part by one skilled in the art. It is necessarily mathematically homogeneous and completely lossless with respect to the totality of the visual representation, such that any visually expressed configuration of ideas can be captured entirely in the XML-based representation, stored during a hiatus of the Composition Studio 120 by the developer, and subsequently reproduced exactly upon restoration, through any number of such cycles. The detailed elements of the XML-based textual representation part of Language 210 also enable and simplify the various algorithmic transformations which are needed to turn the developer's expression into a running Application 101 and which are embodied in various modules throughout Application Factory System 100.

Thus, for the examples presented here, Visual and XML-Based Representation Language 210 underlies and interlaces all the modules of Composition Studio, as the general form in which are expressed all the various aspects of any Device Application 101 or Device Application Component 102.

To enable the expression of a developer's ideas using Language 210, Composition Studio 120 incorporates Visual Programming Design Toolkit 220 and Multimedia User Interface (UI) Design Toolkit 230 as major modules. Each of these modules supports placement of elements from various palettes onto a corresponding canvas, following a metaphor derived from the practice of the art, and used widely in computer software applications intended for expression of various types of creative ideas due to its simplicity.

Within Visual Programming Design Toolkit 220, Data and Logic Canvas 229 offers a multidimensional placeholder for the expression of data, data flows, data manipulation logic, events, event response logic, workflow order, and other related aspects of a particular Application 101 or Application Component 102 under development. Data and Logic Canvas 229 may be configured to appear as a single workspace, or as multiple linked workspaces, according to the needs and preferences of the particular developer as well as the complexity of the ideas being expressed. Elements may be arranged within the workspaces of Canvas 229 to form scripts that specify behavior, layouts that specify data structure, networks that specify data flow or workflow, and so forth as will be readily apparent to those skilled in the art. Formalized advanced concepts, such as entity relationship diagrams, state transition diagrams, message sequence charts, and others, may be incorporated as well.

In general, specific logic and data elements are dragged onto Canvas 229 from various palettes, and then specific attributes are established through controls such as menus, buttons, and pointer gestures. For example, upon dragging a graphical icon representing a control loop logic element onto Canvas 229, the developer may click the pointer on a box in the icon and then type digits indicating the number of times to repeat the loop. The developer may then drag additional logic elements onto the control loop logic element to establish the behavior to execute repeatedly inside the loop. As another example, the developer may drag a graphical icon representing a data input source such as a keyboard onto Canvas 229, followed by another graphical icon representing a data output destination such as a Short Message Service (SMS) recipient. The developer might then click the pointer in a box on the SMS icon, then type a phone number to identify the destination of a message. The developer might further click the pointer on an outward-directed arrow icon within the keyboard icon, drag the pointer over to an inward-directed arrow on the SMS icon, and release the pointer, thereby expressing that what the end user of this Application 101 types will be sent via SMS to the destination. These are but two examples, but it will be plain to those skilled in the art that innumerable expressions would be possible using various combinations of elements on such a Canvas 229.

The elements that may be placed on Data and Logic Canvas 229 come from a variety of palettes within Visual Programming Design Toolkit 220. Event Palette 221 offers icons representing asynchronous occurrences such as, without limitation, application activation and shutdown or key presses, as well as programmable event-generating elements such as menus and buttons, any of which can be used as script starting points. Logic Palette 222 offers icons representing control structures such as, without limitation, the well known IF/THEN/ELSE, DO/WHILE, FOR/DO, and SELECT, as well as commands such as DISPLAY SCREEN or STOP/EXIT. Data Palette 223 offers icons representing data structures such as, without limitation, numbers, character strings, lists, arrays, and compound objects. Data Palette 223 may also offer tools supporting the advanced elements described above, such as flow and relationship connectors. Math Palette 224 offers icons representing mathematical operations, such as, without limitation, the usual arithmetic operators found on a calculator, Boolean operators, and complex functions such as might be seen in a standard spreadsheet program. Service Palette 225 offers icons representing specific features of a device, such as its communication and sensing capabilities. In a sense, Service Palette 225 embodies visually what would in a traditional programming environment be called the Application Programming Interface (API) for specific device functions. Example elements to be found in Service Palette 225, representing capabilities typically available in most devices, include without limitation, communication channel tools that provide access to information transfer services such as SMS and MIMS, E-mail, RSS, Telephony, Wifi and Bluetooth links, various Instant Messenger (IM) and Voice over Internet Protocol (VoIP) clients such as GTalk or Skype, and various complex clients for social network services such as Facebook or Twitter; location information tools such as a GPS receiver or location sensor, an accelerometer or motion sensor, and interactive map databases; time and date tools such as a clock, a calendar, and various alarms; and information/productivity tools such as a memo pad or personal note composer and database, a sketch pad or personal drawing composer and database, an address book or contact list database, an agenda or personal calendar event database, and a conversation record or message composer and database. Specific functions and data offered by each of the Service Palette 225 elements listed above is dependent on the service provided, but will be readily evident to one skilled in the art upon reflection, investigation of the cited services, and examination of typical feature sets found in devices on the market today. Finally, Workflow Palette 226 offers icons representing BPMN workflow elements, which are described more fully in the context of FIG. 3.

Within Multimedia UI Design Toolkit 230, Multimedia UI Canvas 239 offers a multidimensional placeholder for the expression of screen presentations, audio input and output, text input and output, and other related user interaction aspects of a particular Application 101 or Application Component 102 under development. Multimedia UI Canvas 239 may be configured to appear as a single workspace, or as multiple linked workspaces, according to the needs and preferences of the particular developer as well as the complexity of the ideas being expressed. Elements may be arranged within the workspaces of Multimedia UI Canvas 239 to form screen layouts, animations, and so forth as will be readily apparent to those skilled in the art.

In general, specific user interaction elements are dragged onto Multimedia UI Canvas 239 from various palettes, and then specific attributes are established through controls such as menus, buttons, and pointer gestures. For example, upon dragging a graphical icon representing a new screen element onto Multimedia UI Canvas 239, the developer may then drag additional graphic elements onto the screen element to establish the visual content of that screen, and use a menu to assign an animation behavior to one or more of the graphic objects on that screen. This is but one example, but it will be plain to those skilled in the art that innumerable expressions would be possible using various combinations of elements on such a Multimedia UI Canvas 239.

The elements that may be placed on Multimedia UI Canvas 239 come from a variety of palettes within Multimedia UI Design Toolkit 230. Graphic Palette 231 offers icons representing graphic objects to be drawn on the Multimedia UI Canvas 239, such as, without limitation, line segments, polygons, complex predefined shapes, and freehand shapes. Widget Palette 232 offers icons representing information input and output, including without limitation buttons, text boxes, list boxes, drop-down lists, and so forth. Audio/Video Palette 233 offers icons representing sounds, movies, photos, or other audible and visible entities that may be detected (input) or played (output), and tools for manipulating or altering such items that may be produced or encountered within an Application 101. Examples of manipulation or alteration elements that may be offered on Audio/Video Palette 233 include, without limitation, a speech recognition capability or speech to text translator, and a text reading capability or text to speech translator. Haptic Palette 234 offers icons representing mechanical vibrations, feedback forces, or gestures that may be detected or played, and tools for manipulating or altering such items that may be produced or encountered within an Application 101.

As previously mentioned in the context of FIG. 1, a developer may choose to target a Specific Device Model 104 for a particular Application 101 or Application Component 102, rather than developing for the Generic Device Model 103. This choice is expressed in Composition Studio 120 through Specific Device Selector 214. In an exemplary embodiment, Specific Device Selection 214 may present to the developer a list of supported Specific Device Models 104, which may include both individual devices and device families or brands, and web platform language, and allow one or more to be selected. When a Specific Device Model 104 is selected as the target for a particular development project for a particular Application 101 or Application Component 102, the items available in the various Palettes of Toolkits 220 and 230 are constrained such that primarily those pertinent to features present in the Specific Device Model 104 selected via Specific Device Selector 214 may be used. For example, if a selected Specific Device Model 104 does not have a motion sensor or accelerometer, the corresponding capabilities would be disabled or not even visible within Service Palette 225. Similarly, if a selected Specific Device Model 104 does not have the ability to apply vibrations or force feedback in response to touch-screen events, the corresponding capabilities would be disabled or invisible within Haptic Palette 234. Other constraints will be readily evident, following from this principle, to one skilled in the art.

Visual Programming Design Toolkit 220 and Multimedia UI Design Toolkit 230 are interconnected within Composition Studio 120 such that elements assigned to the Canvas of one may have related manifestations in the Canvas of the other. For example, if a new screen element is created within Multimedia UI Canvas 239, it will become possible for the DISPLAY SCREEN command in Logic Palette 222 to identify the new screen element as the one to be displayed. If a programmable button element is created on Data and Logic Canvas 229, it will become possible to link it with a graphic element on a screen on Multimedia UI Canvas 239, such that when an end user of the corresponding Application 101 points at that area of the screen, the programmed event is triggered. More complex examples can be constructed using the capabilities of Composition Studio 120, as will be readily apparent to those skilled in the art.

In an exemplary embodiment, all palettes within both Design Toolkits 220 and 230 are designed to be extensible so that elements can be added and changed easily as the Language 210 evolves. New palettes may be defined as well, such as the new Workflow Palette 226 added in the present disclosure. Further, each palette can be extended to incorporate Application Components 102, and even embeddable Applications 101, of the corresponding semantic class. Design Toolkits 220 and 230 may discover automatically, or support developers in their interactive discovery of, Application Components 102 and embeddable Applications 101 that are available in Distribution Center 130, including in particular previously coded workflows that can be reused via the new Workflow Palette 226. Distribution Center Library Client 215 provides the mechanism implementing the Composition Studio 120 end of Service Interaction 132 with Distribution Center 130, in support of this capability.

Default palette elements, as well as discovered Application Components 102 and embeddable Applications 101 retrieved from Distribution Center 130 by Distribution Center Library Client 215, are cached within local repositories at Composition Studio 120 for use by Design Toolkits 220 and 230. Default multimedia elements and semantically related Application Components 102 are kept in UI Object Library Cache 213. Default logic and data elements, including event, math, and service elements, and semantically related Application Components 102, are kept in Service Object Library Cache 212. Applications 101 that can be incorporated whole into other Applications 101, or which include pieces which may be incorporated separately from the whole, are kept in Embeddable App Library Cache 211. In an exemplary embodiment, Library Caches 211, 212, and 213 are implemented as separate database instances of identical structure for simplicity.

When a developer has expressed enough ideas to form something usable as an application or application component, it should be tested using the capabilities of Visual Verification Toolkit 240. The elements on Data and Logic Canvas 229 can be executed, and the elements on Multimedia UI Canvas 239 can be presented, individually or together as a whole according to the preferences and needs of the developer, in a high fidelity simulation environment essentially identical to that which will exist on any target Mobile Device 181 or 191 or any target web platform.

Locally within Composition Studio 120, Visual Verification Toolkit 240 provides testing in the environment of the Generic Device Model 103. The portion of Generic Device Model 103 that is relevant for testing, in particular the runtime executive but not the compilation tools, is incorporated in Visual Verification Toolkit 240 as Generic Device Model Testing Module 241. In addition, so that the developer may interactively observe the execution of expressed elements, two additional modules are desirable.

Generic Device Model Interface Simulator 242 provides linkages between physical devices on the developer's Computer 160 or 170, and logical devices of Generic Device Model Testing Module 241. For example, Computers 160 and 170 are likely to have a microphone and speakers, perhaps a camera, and certainly a screen and keyboard. Interface Simulator 242 links these devices with corresponding devices in the Generic Device Model Testing Module 241. Most Computers 160 and 170 are likely to be able to play arbitrary sound files and video files as well, allowing Interface Simulator 242 to superimpose those media streams on corresponding simulated inputs and outputs as appropriate for testing, or in place of corresponding real input or output devices. Most Computers 160 and 170 will have a touchpad or a mouse, either of which can be mapped by Interface Simulator 242 onto a touchpad of Generic Device Model Testing Module 241. Some Computers 160 or 170 may have a multitouch pad or screen, which Interface Simulator 242 can map onto a multitouch pad of Generic Device Model Testing Module 241. Most Computers 160 and 170 will have Internet connections, and many will have access to Internet-based email, instant messaging, and voice calling services. Interface Simulator 242 can map these services onto wireless communication links and corresponding communication service interfaces within Generic Device Model Testing Module 241.

Generic Device Model Capability Simulator 243 provides simulations of certain Generic Device Model Testing Module 241 capabilities that would not normally be available on any Computer 160 or 170. For example, the GPS receiver and accelerometer of many real mobile devices, which are modeled in Generic Device Model Testing Module 241, have no physical counterpart on the typical Computer 160 or 170. Therefore, Capability Simulator 243 may provide a visual simulation of location, whereby an icon representing the modeled mobile device is shown superimposed on a map at a developer-selected location, and Generic Device Model Testing Module 241 is given inputs such that its simulated GPS receiver reports the selected location to logic and data elements using it. The developer may click on the device icon and drag it to a new location, or select a mode in which the simulated device is propagated along a selected path at a particular velocity, and the simulated GPS receiver will report, again to those logic and data elements using it, the location as it changes while moving. Similarly, a fine-scale motion simulation may be provided by Capability Simulator 243, whereby the developer may select a mode similar to a three-dimensional fly-around display in which the simulated device may be shaken or placed in different orientations by clicking and moving the mouse of Computer 160 or 170. In this mode, the simulated accelerometer in Generic Device Model Testing Module 241 will report, to those logic and data elements using it, corresponding fine motion changes.

In some situations, the developer will not be satisfied with testing against only the Generic Device Model Testing Module 241. If an Application 101 under development relies upon unique features of a Specific Device Model 104, or if for any reason the developer wishes to be absolutely certain of the behavior on a particular model, Visual Verification Toolkit 241 has the ability to access any and all Specific Device Models 104 for testing. This is accomplished by utilizing Verification Engine 150 through Verification Engine Client 245, which implements the Composition Studio 120 end of Service Interaction 152. Prior to executing the Application 101 under development on a real Mobile Device 191 or Specific Device Model 104 or real web platform, it will be desirable to compile the expressed design into an actual executable suitable for the target. This is accomplished by Build Engine 140, through Build Engine Client 244, which implements the Composition Studio 120 end of Service Interaction 142.

In general, a complete and tested Application 101 or Application Component 102 may be submitted to Distribution Center 130 for consideration. Distribution Center Submission Client 216 provides the ability to do so, implementing the submission aspects of the Composition Studio 120 end of Service Interaction 132. Relevant aspects include, without limitation, identifying and transmitting the submitted Application 101, identifying the submitting developer as a specific user of Distribution Center 130, and setting a price (which may be zero) for others to use it.

A thorough developer will generally build and test the completed Application 101 or Application Component 102 on multiple real Devices 191, Specific Device Models 104, and web platforms. To ease this process, Build Engine Client 244 and Verification Engine Client 245 provide the ability to submit the Application 101 or Application Component 102, as expressed in Language 210, to Build Engine 140 for builds on multiple Specific Device Models 104, and then to Verification Engine 150 for automatic verification using a detailed test plan on multiple, or even all, Specific Device Models 104. More information on these bulk build and verification modes is provided in U.S. Pat. No. 8,719,776's FIGS. 4 and 5, and attendant description, as well as in the incorporated applications listed above.

Tying together all the user-facing functionality of Composition Studio 120 is Graphical User Interface 260. This is a design module that provides graphical and interactional support for each of the functions already described. It is implemented, using techniques well known to those skilled in the art, in a manner that integrates with the functional modules and relies upon the features of User Interface Support Module 202. An example layout that may be used for User Interface 260 in an exemplary embodiment is presented in U.S. Pat. No. 8,719,776's FIG. 6 and attendant description, as well as in the incorporated applications listed above, and in FIGS. 5 and 6 below.

One of ordinary skill in the art will recognize that system 100 and its elements may be implemented in various different ways without departing from the scope of the disclosure. For instance, the various elements of the system may be arranged in various different ways. As another example, various devices or elements may be implemented using multiple devices or sub-elements. Likewise, in some embodiments multiple devices or elements may be combined into a single device or element. In addition, various other elements may be included and/or various listed elements may be omitted in some embodiments.

FIG. 3 illustrates an exemplary workflow and corresponding exemplary user interface screens which may be included in an Application 101 or Application Component 102. Example workflow 380 may be created using the Workflow Palette 226 and other tools in Visual Programming Design Toolkit 220. Similarly, example user interface screens 390 may be created using the tools in Multimedia UI Design Toolkit 230. Note that the exact nature of the workflow shown in FIG. 3 is a simple patient questionnaire that might be created in the SnapClinical instantiation of Application Factory System 100, for example, a clinical trial and so forth. However, it is described here in terms of its logical and structural symbology in order to focus on the capability of building relevant workflows rather than on any specific workflow topic. A developer may create any number of such workflows and user interfaces as needed for a particular Application 101 or Application Component 102.

The representation of example workflow 380 and its example user interface 390 thus includes a number of symbols with specific meanings in the context of Workflow Palette 226. Workflow label 381 and user interface label 391 distinguish between the logical and graphical specifications, while workflow name 387 and user interface name 397 contain text strings created by the developer to distinguish this workflow and its user interface from others. The same value, “Ask About Symptoms” in the example, is shown for both names 387 and 397 to indicate that this workflow 380 and this user interface 390 are related to one another.

The flow aspect of example workflow 380 begins at start symbol 331 and is complete at end symbol 335. Other symbols not shown may represent waiting for a message from an external entity or suspending for a period of time. To get from start to end, a variety of actions, decisions, and transitions may occur. Each action block 382 provides an opportunity for the application implementing example workflow 380 to do something useful, which typically may be summarized using the action block name 321. The actual details of what the action block 382 does may be specified by invoking a composition studio canvas and palette that corresponds to the action block type. The action block name 321 may also match a named element in the corresponding canvas, so as to specify a definitive relationship between the workflow action block and the underlying action. Each transition 383 may provide a time-ordered flow from a start symbol 331 or action block 382 to another action block 382, an end symbol 335, or a decision block 332. Each decision block 332, of which only one is present in FIG. 3, may provide a branch in the flow based on a data item identified by the preceding action block 382. Each possible value of the data item may then be used as a transition name 334 associated with a corresponding named transition 333, such that the decision block 332 directs the flow along the corresponding path according to the selected value. Note that the only type of decision block 332 shown in FIG. 3 is similar in meaning to a CASE statement in a traditional computer programming language. Other decision block types may be used, each one represented using a different symbol not shown, including such common operations as logical and numerical comparisons, as well as a multipath operation that splits the flow into two or more simultaneous sequences.

Three types of action block are depicted here using distinct icons to represent each, and others may be inferred from the composition studio descriptions above and in the incorporated patent applications and patent, which together describe more than this number of canvas/palette combinations. Action block type 322 may manipulate data, such as reading a variable in the type 322 action block 382 labeled “Pick Question Style” or writing a variable in the type 322 action block 382 labeled “Record Response.” Within Composition Studio 120, selecting or creating an action block 382 of type 322 may invoke the data palette of the data and logic canvas in order to specify the pertinent data items and what is to be done with them. It should be noted that multiple “types” of data manipulation can be performed by the composition studio, the types and action coming from linked or incorporated toolkits, non-limiting examples being multimedia user interface design and programming design toolkits, additional examples being found in the aforementioned antecedents.

Action block type 323 may cause the presentation of a corresponding screen or subscreen according to the relationship indicated via the action block name 321. Within Composition Studio 120, selecting or creating an action block 382 of type 323 may invoke the user interface canvas to specify the corresponding user interface elements. More detail regarding the construction of corresponding screens follows in the context of example user interface 390.

Action block type 324 may cause the execution of an algorithm, effectively a subroutine that matches the action block name 321. Within Composition Studio 120, selecting or creating an action block 382 of type 324 may invoke the logic palette of the data and logic canvas in order to specify the algorithm to be executed. Any operation that may be expressed in the data and logic canvas may be incorporated in the corresponding algorithm; thus an action block 382 of type 324 may perform calculations, interact with other entities through external communication, start or stop sensors or actuators in the device running Workflow 380 in the context of an Application 101 or Application Component 102, or any other operation available in that portion of the composition studio. This palette and canvas are not further described here; the aforementioned antecedents provide details.

As previously noted, example user interface 390 is associated with example workflow 380 through the common value in their respective names 387 and 397. In the user interface canvas, a developer may arrange passive graphical elements such as text 343 and icons 344, and active graphical elements such as selectors 345 and buttons 346. These arrangements may be grouped into fixed areas such as header 341 and footer 342 so that they appear in every variation of screen 394, or they may be grouped into subscreens 353 linked to a variable area 395 by subscreen relationships 351 so that the appropriate visual and control arrangement may be selected according to workflow logic. Subscreen names 352 would then be used to associate each subscreen 353 with an action block of type 323 by matching its action block name 321.

One skilled in the art will appreciate that the fixed areas header 341 and footer 342 in screen 394 may have as easily been designed using additional variable areas 395 and accompanied by additional subscreens 353 with corresponding name relationships 352 to additional action blocks 382 of type 323. Further, the specific decisions regarding structure and design of a workflow and its user interface are entirely at the developer's discretion and may take any conceivable form, including more or fewer elements in different orders with simpler or more complex logic. Similarly, the values of names 387, 397, 321, 334, and 352, as well as the content and form of graphical elements such as text fields 343, images 344, and buttons 345 and 346, are also design choices that may be exercised by the developer. One skilled in the art will also appreciate that the range of logic and graphic elements is not limited to those described here. The full set of capabilities supported by Composition Studio 120 may be utilized in designing real workflows and user interfaces for a specific Application 101 or Application Component 102. Further, it should be noted that the specific graphic style of each symbol depicted in FIG. 3 is important only to the extent that it evokes the workflow or user interface element it is meant to represent. Other symbols and symbol positions may be used equally as well within the scope of the present disclosure.

FIG. 4 illustrates an exemplary workflow which may be created using Composition Studio 120 and included in an Application 101 or Application Component 102 targeted for a web platform or mobile device using the capabilities of Application Factory System 100. Example work flow 8801 depicts what may be a structure for a participant qualification workflow, wherein a patient candidate may determine whether he or she is a fit for a specific clinical trial supported by a particular instance of system 100 as it might be applied in SnapClinical. Note that the associated user interface screens for example workflow 8801 are not depicted, but would certainly exist, having been created using the visual language described in the context of example user interface 390 in FIG. 3. One skilled in the art will recognize the visual language of example workflow 8801 from its earlier explanation using example workflow 380 in FIG. 3, so the detailed flow is not described further other than to point out that example workflow 8801 has a richer structure with more decision points than example workflow 380.

FIG. 5 depicts a snapshot of an exemplary screen view of Workflow Palette 226 and Data and Logic Canvas 229 in the Composition Studio 120 operating under Visual Programming Design Toolkit 220. Exemplary Workflow View 5000 works with the above modules to provide placement of elements from exemplary Workflow Palette 5226 onto a corresponding exemplary Canvas 5229. Elements may be arranged within the workspaces of exemplary Canvas 5229 to form scripts that specify behavior, layouts that specify data structure, networks that specify data flow, and so forth as will be readily apparent to those skilled in the art. For example, FIG. 5 shows specific logic control and data decision elements that are “dragged” from the exemplary Workflow Palette 5226 suite onto Canvas 5229, to feed into other pre-existing process(es), or to start the first process (e.g., Start Events). The methodology of binding processes together to form a control and decision workflow path may be through “snapping” together adjacent designator icons/elements, or via a selection process, grouping, and so forth. U.S. Pat. No. 8,719,776, for example, contains detailed descriptions and examples of such methodologies, variations of which are understood to be within the purview of this disclosure.

An assortment of available workflow elements, such as Start Events, Activities, Structural, Gateways, Boundary Events, Intermediate Catching Events, Intermediate Throwing Events, End Events, Swimlanes, and Artifacts are displayed in the exemplary Workflow Palette 5226, understanding the list may be varied with other choices, submenus, etc. according to design preference. Exemplary Canvas 5229 shows various workflow elements that have been placed and connected to define a specific workflow that might form part of an Application 101 or Application Component 102 associated with a clinical trial by a SnapClinical instantiation of system 100. The nature of the elements shown have been defined in the context of FIG. 3 above. Other workflow elements not shown but available in exemplary Workflow Palette 5226 may be as defined in a workflow language standard such as BPMN.

Commensurate with the exemplary Workflow Palette 5226, the exemplary software contains a menu bar 5500 providing additional workflow palette categories, for example, Case models, Forms, Decision Tables, Apps, and so forth. Each option may be selected to cause a different exemplary Workflow Palette 5226 to be displayed and the associated “drag and snap” of the palette items in the sub-menu can be loaded into the exemplary Canvas 5229. By selecting the desired category from the menu bar 5500, and then selecting the desired operation or element from the respective palette, a decision workflow with associated pathways and data in/out conditions can be developed using simple “drag and snap” procedures. No coding is needed. No expertise in software is needed.

FIG. 6 is a snapshot of an exemplary screen view of Graphic Palette 231 and Multimedia UI Canvas 239 in the Composition Studio 120 operating under Multimedia User Interface (UI) Design Toolkit 230. Exemplary Design View 6000 works with the above modules (and optionally in conjunction with Exemplary Workflow View 5000) to provide a build of user interface windows or interactions. It is noted here, that a process built with the tools in Exemplary Workflow View 5000 may not require a user interface for its data in/out actions, relying instead on database input, etc. However, in the example of FIG. 6, the Exemplary Design View 6000 is illustrated as providing the age check UI's for the age check process(es) depicted in Exemplary Workflow View 5000 of FIG. 5.

The Exemplary Design View 6000 UI composition steps are in many ways similar to the composition steps found in the Exemplary Workflow View 5000. For example, a Canvas 6239 is on the desktop wherein a list of pre-configured UI operations, shapes, actions, containers, etc. are presented in Palette 6231. One or more selections of which can be “dragged and dropped” into the Canvas 6239 to form a set of UI windows that may be linked to the action blocks chosen for Exemplary Workflow View 5000 in FIG. 5. As stated above, other composition actions to form the UIs may include “dragging and dropping/snapping,” or a selection process, grouping, and so forth. U.S. Pat. No. 8,719,776, for example, contains detailed descriptions and examples of such methodologies, variations of which are understood to be within the purview of this disclosure.

The UI being composed can be associated to a specific logic control and data decision element via Navigator 6300, which may take the form of a directory tree or a smaller representation of the workflow exemplified in FIG. 5, wherein common naming as described in FIG. 3 links the objects in Canvas 5229 to those in Canvas 6239. Size, shape, color, and other assorted attributes of a screen as well as the text and objects on it may be customized via customization pane 6400. Finally, a menu bar 6500 provides additional controls, which may include zooming in and out on the view, switching between the logic and visual design views (that is, between that of FIG. 5 and that of FIG. 6), and—in the highlighted area—preparing the Application 101 or Application Component 102 for deployment. The highlighted menus represent Specific Device Models 104, and are named according to dominant platform types at the time of this writing, but may at other times feature other platform names as appropriate. Each menu may contain commands for verifying, building, and deploying the Application 101 or Application Component 102 to the selected Specific Device Model 104 named in the menu. The commands in these menus thereby embody linkages between Composition Studio 120 and the other major elements of Application Factory System 100, namely Build Engine 140, Verification Engine 150, and Distribution Center 130.

As stated above, the tools shown in the exemplary workflow and visual design views are able to assist in creating not only native mobile applications for deployment to all existing mobile device types as previously disclosed, but also web applications for deployment to a web browser via a web server. Each application may be deployed as either a web application or a mobile application, or both, at the discretion of the developer using the Application Factory system. Additionally, as seen above, the Visual Representation Language has been extended to incorporate multiple additional modes of expression covering multiple additional types of logic and data structure. The Composition Studio is able to support these new language modes, and the Build Engine is now able to incorporate corresponding code into generated applications, services, and data structures. The Application Factory system's Composition Studio provides a workflow palette of BPMN model elements and a workflow canvas on which to arrange them, while its Build Engine provides automatic translation of BPMN workflows into its internal XML-Based Representation Language and thence to compiled native mobile device (IOS, Android, etc.) and web (HTML5) applications.

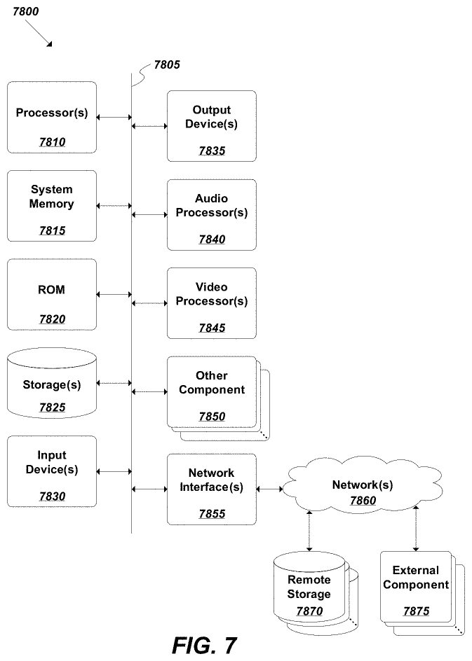

FIG. 7 illustrates a schematic block diagram of an exemplary computer system 7800 used to implement the exemplary embodiments. For example, the systems described above in reference to FIGS. 1-6 may be at least partially implemented using one of more instances of computer system 7800.

Computer system 7800 may be implemented using various appropriate devices. For instance, the computer system may be implemented using one or more personal computers (PCs), servers, mobile devices (e.g., a smartphone), tablet devices, and/or any other appropriate devices. The various devices may work alone (e.g., the computer system may be implemented as a single PC) or in conjunction (e.g., some components of the computer system may be provided by a mobile device while other components are provided by a tablet device).

As shown, computer system 7800 may include at least one communication bus 7805, one or more processors 7810, a system memory 7815, a read-only memory (ROM) 7820, permanent storage devices 7825, input devices 7830, output devices 7835, audio processors 7840, video processors 7845, various other components 7850, and one or more network interfaces 7855.

Bus 7805 represents all communication pathways among the elements of computer system 7800. Such pathways may include wired, wireless, optical, and/or other appropriate communication pathways. For example, input devices 7830 and/or output devices 7835 may be coupled to the system 7800 using a wireless connection protocol or system.

The processor 7810 may, in order to execute the processes of some embodiments, retrieve instructions to execute and/or data to process from components such as system memory 7815, ROM 7820, and permanent storage device 7825. Such instructions and data may be passed over bus 7805.

System memory 7815 may be a volatile read-and-write memory, such as a random access memory (RAM). The system memory may store some of the instructions and data that the processor uses at runtime. The sets of instructions and/or data used to implement some embodiments may be stored in the system memory 7815, the permanent storage device 7825, and/or the read-only memory 7820. ROM 7820 may store static data and instructions that may be used by processor 7810 and/or other elements of the computer system.

Permanent storage device 7825 may be a read-and-write memory device. The permanent storage device may be a non-volatile memory unit that stores instructions and data even when computer system 7800 is off or unpowered. Computer system 7800 may use a removable storage device and/or a remote storage device as the permanent storage device.

Input devices 7830 may enable a user to communicate information to the computer system and/or manipulate various operations of the system. The input devices may include keyboards, cursor control devices, audio input devices and/or video input devices. Output devices 7835 may include printers, displays, audio devices, etc. Some or all of the input and/or output devices may be wirelessly or optically connected to the computer system 7800.

Audio processor 7840 may process and/or generate audio data and/or instructions. The audio processor may be able to receive audio data from an input device 7830 such as a microphone. The audio processor 7840 may be able to provide audio data to output devices 7840 such as a set of speakers. The audio data may include digital information and/or analog signals. The audio processor 7840 may be able to analyze and/or otherwise evaluate audio data (e.g., by determining qualities such as signal to noise ratio, dynamic range, etc.). In addition, the audio processor may perform various audio processing functions (e.g., equalization, compression, etc.).

The video processor 7845 (or graphics processing unit) may process and/or generate video data and/or instructions. The video processor may be able to receive video data from an input device 7830 such as a camera. The video processor 7845 may be able to provide video data to an output device 7840 such as a display. The video data may include digital information and/or analog signals. The video processor 7845 may be able to analyze and/or otherwise evaluate video data (e.g., by determining qualities such as resolution, frame rate, etc.). In addition, the video processor may perform various video processing functions (e.g., contrast adjustment or normalization, color adjustment, etc.). Furthermore, the video processor may be able to render graphic elements and/or video.

Other components 7850 may perform various other functions including providing storage, interfacing with external systems or components, etc. Finally, as shown in FIG. 7, computer system 7800 may include one or more network interfaces 7855 that are able to connect to one or more networks 7860. For example, computer system 7800 may be coupled to a web server on the Internet such that a web browser executing on computer system 7800 may interact with the web server as a user interacts with an interface that operates in the web browser. Computer system 7800 may be able to access one or more remote storages 7870 and one or more external components 7875 through the network interface 7855 and network 7860. The network interface(s) 7855 may include one or more application programming interfaces (APIs) that may allow the computer system 7800 to access remote systems and/or storages and also may allow remote systems and/or storages to access computer system 7800 (or elements thereof).

Many of the processes and modules described above may be implemented as software processes that are specified as one or more sets of instructions recorded on a non-transitory storage medium. When these instructions are executed by one or more computational element(s) (e.g., microprocessors, microcontrollers, digital signal processors (DSPs), application-specific integrated circuits (ASICs), field programmable gate arrays (FPGAs), etc.) the instructions cause the computational element(s) to perform actions specified in the instructions.

In some embodiments, various processes and modules described above may be implemented completely using electronic circuitry that may include various sets of devices or elements (e.g., sensors, logic gates, analog to digital converters, digital to analog converters, comparators, etc.). Such circuitry may be able to perform functions and/or features that may be associated with various software elements described throughout.

As used in this specification and any claims of this application, the terms “computer”, “server”, “processor”, and “memory” all refer to electronic devices. These terms exclude people or groups of people. As used in this specification and any claims of this application, the term “non-transitory storage medium” is entirely restricted to tangible, physical objects that store information in a form that is readable by electronic devices. These terms exclude any wireless or other ephemeral signals.

It should be recognized by one of ordinary skill in the art that any or all of the components of computer system 7800 may be used in conjunction with some embodiments. Moreover, one of ordinary skill in the art will appreciate that many other system configurations may also be used in conjunction with some embodiments or components of some embodiments.

In addition, while the examples shown may illustrate many individual modules as separate elements, one of ordinary skill in the art would recognize that these modules may be combined into a single functional block or element. One of ordinary skill in the art would also recognize that a single module may be divided into multiple modules.

The foregoing relates to illustrative details of exemplary embodiments and modifications may be made without departing from the scope of the disclosure as defined by the following claims.

What is claimed is:

1. An Application Factory system containing computer-executable instructions, comprising:
a Composition Studio, where a workflow for at least one of a Device Application and a Device Application Component is generated from a series of user-initiated selections of at least one of a logic and data element from an Event, Logic, Data, Math, Workflow, or Service Palette, and a user interaction element from a Graphic, Widget, Audio/Video, or Haptic Palette, the workflow being displayed on at least one of a Data and Logic Canvas and Multimedia User Interaction Canvas of a user device;
a Build Engine supporting a compilation of the Device Application or Device Application Component;
a Generic Device Model providing a test of the Device Application or Device Application Component; and
a Distribution Center, running on a server, providing at least one of discovery of, education on, demonstration of, experimentation with, and acquisition of the Device Application or Device Application Component,
wherein the Composition Studio utilizes a Visual and XML-Based Representation Language to enable the workflow to be generated without the user having to perform software coding.

2. The system of claim 1, further comprising a Verification Engine providing step level simulation and testing of the Device Application or Device Application Component.

3. The system of claim 1, further comprising a selection option in the Composition Studio, the option enabling the Build Engine to compile the Device Application or Device Application Component for operation on either a mobile device or web browser.

4. The system of claim 2, wherein the workflow is a clinical trial workflow.

5. The system of claim 1, further comprising a computer running the Application Factory system's computer-executable instructions.

6. The system of claim 1, further comprising an Internet connection between the user device and the Distribution Center's server.

7. The system of claim 1, further comprising an Internet connection between the mobile device or web browser and the Distribution Center's server.

8. A method for building and distributing a device software application without software coding, comprising computer-implemented steps of:
generating a workflow for at least one of a Device Application and a Device Application Component from a series of user-initiated selections of at least one of a logic and data element from an Event, Logic, Data, Math, Workflow, or Service Palette, and a user interaction element from a Graphic, Widget, Audio/Video, or Haptic Palette;
displaying the workflow on at least one of a Data and Logic Canvas and Multimedia User Interaction Canvas on a user device;
compiling the Device Application or Device Application Component for operation on at least one of a mobile device and web browser;
testing of the Device Application or Device Application Component; and
uploading the Device Application or Device Application Component to a Distribution Center, wherein the Device Application or Device Application Component can be run on the mobile device or web browser,
wherein a Visual and XML-Based Representation Language is used to enable the workflow to be generated without performing software coding.

9. The method of claim 8, further comprising simulating and testing of the Device Application or Device Application Component at a step level.

10. The method of claim 8, further offering a selection option to compile the Device Application or Device Application Component for operation on either a mobile device or web browser.

11. The method of claim 8, wherein the generated workflow is a clinical trial workflow.
